(19) United States
(12) Plant Patent Application Publication
(10) Pub. No.: US 2008/0141393 P1
(43) Pub. Date: Jun. 12, 2008

(54) Variety of rose plant named 'GLE01'

(76) Inventor: Robin Somerfield, Tauranga (NZ)

Correspondence Address: FULWIDER PATTON LLP, 200 OCEANGATE, SUITE 1550, LONG BEACH, CA, 90802, US

(21) Appl. No.: 11/894,523

(22) Filed: Aug. 21, 2007

(30) Foreign Application Priority Data
Nov. 8, 2006 (NZ) ROS917

(51) Int. Cl. A01H 5/00

(52) U.S. Cl. PLT/103

(57) ABSTRACT

A new and distinct cultivar of Rose plant named GLE01’ is characterized in that it is more compact with higher number of flowers when compared with its closest comparator (‘ICEBERG’) and includes a white flower and dark green leaf color. The habit of ‘GLE01’ is compact as opposed to spreading, and it possesses a larger flower size along with an extended and earlier flowering season. It possesses a light and pleasing fragrance and exhibits very good disease resistance.

### BACKGROUND OF THE NEW PLANT

The present invention relates to a new and distinct cultivar of Rose, botanically known as Rosa and further known by the varietal name ‘GLE01.’ This new variety is also sold under the Breeder's reference name SOMHITMAS. The new variety was propagated via controlled pollination of Rosa Anniversary and Rosa Sky Tower. The seedlings were first selected in 1998, and initially increased by budding and then subsequently by cutting. The seeds were planted out and the plants with desirable ornamental characteristics were selected. ‘GLE01’ was selected for its foliage color, flower habit, growth habit, fragrance. Thie cultivar was isolated by the inventor, Robin Somerfield, in Tauranga, New Zealand. Cuttings were taken from the selected plant and were grown on to assure stability. Plants were grown over many generations and all have been stable. Propagation by cuttings in other locations have also proved stable, and the desirable features of the plant have remained constant throughout the evaluation period.

### SUMMARY OF THE INVENTION

Plants of the new Rose variety GLE01 can be compared to other plants of its genus, the closest comparator being ICEBERG. Plants of the cultivar ‘GLE01’ are denser and more compact when compared with ICEBERG. GLE01 possesses an erect, upright flower as opposed to a slightly drooping flower of ICEBERG. Other distinguishing features are presented below:

| Characteristic | Iceberg | GLE 01 |
|---|---|---|
| Petal number | 26 to 32 | 28 to 40 |
| Petal Width | average 30 mm | average 40 mm |
| Petal shape | ovate | broadly ovate |
| Petal color | near white | near white with |
| | | lemon basal |
| | | blotch |
| Buds | Smaller/pointed | Larger/Rounded |
| Hips | Elongated | More rounded |
| Leaves | Longer/pointed | Compact/rounded |
| Flowers per peduncle | 3 to 5 | Up to 10 |
| Pedicle | brown hairs/bristles | Smooth. |

The habit of ‘GLE01’ is compact as opposed to spreading, and it possesses a larger flower number per peduncle, along with a light fragrance, dark green foliage, and very good disease resistance. It is well suited for gardens and outdoor pots.

The foregoing traits have been repeatedly observed and are determined to be among the unique characteristics of ‘GLE01’. These characteristics distinguish ‘GLE01’ as a new and distinct cultivar:

### DESCRIPTION OF THE DRAWING

The accompanying colored photographs illustrate the new cultivar:

FIG. 1 shows an enlarged view of the plant and flowers;

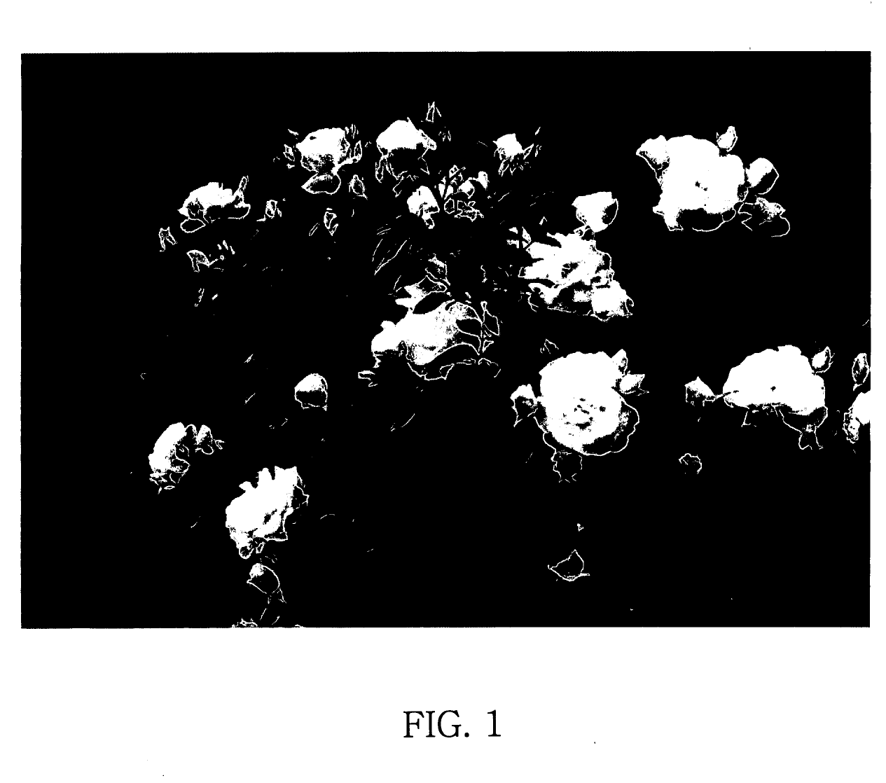

FIG. 2 shows a second enlarged view of the plant and flowers; and

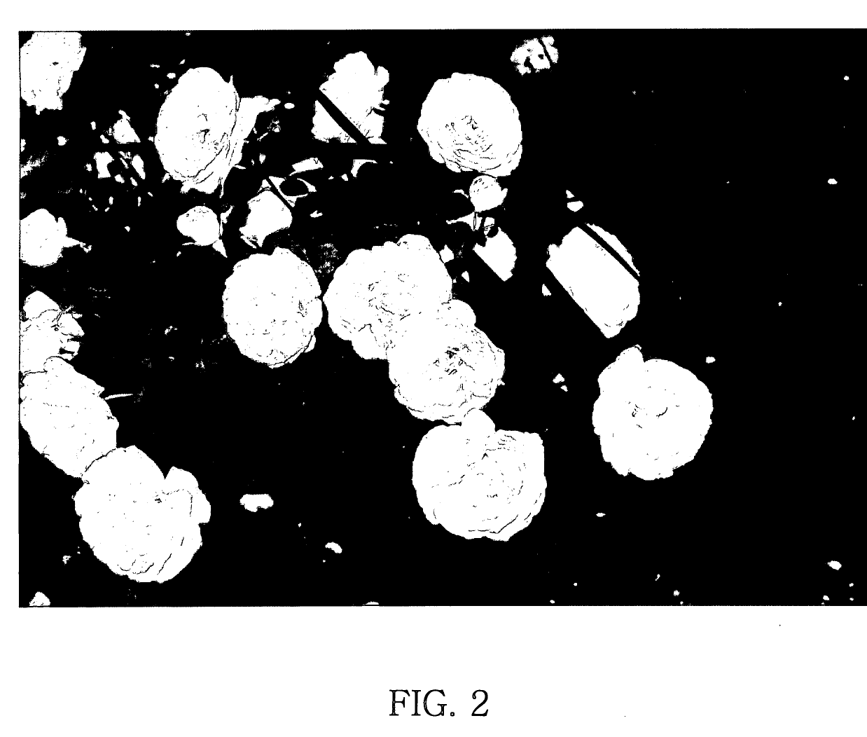

FIG. 3 shows a third enlarged view of the plants and flowers.

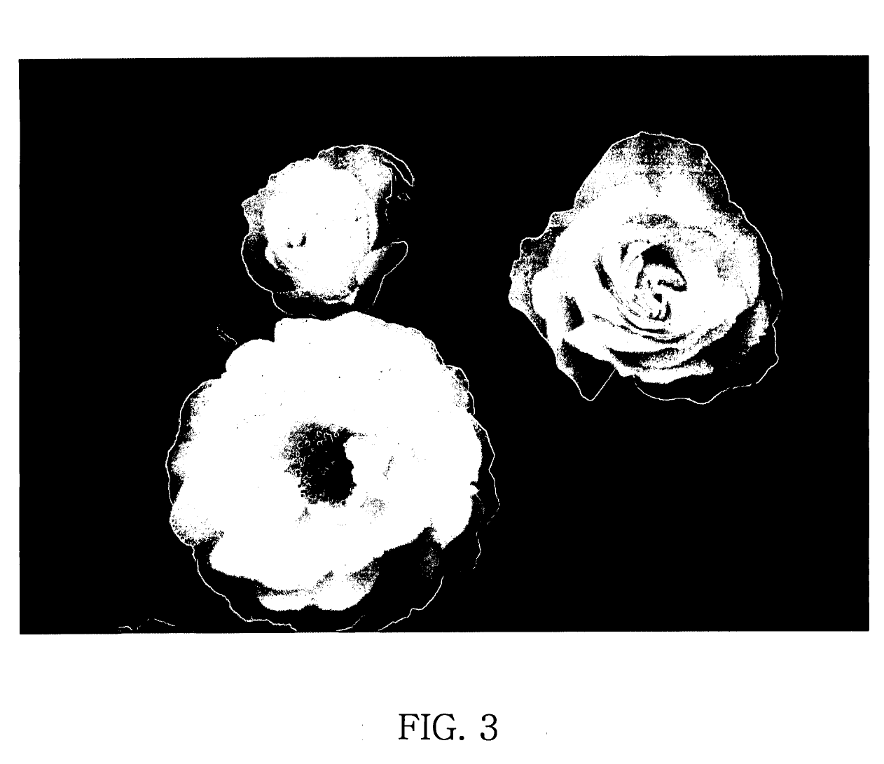

### DETAILED BOTANICAL DESCRIPTION OF THE NEW PLANT

The following detailed description sets forth characteristics of the new cultivar. The data which defines each characteristic was collected by the breeder at Taulalnga, New Zealand.

In the following table of comparative features, color references are made to The Royal Horticultural Society of London Colour Chart, 1995 Edition where available.
Botanical name: Rosa
Varietal name: ‘GLE01’
Parentage: Rosa Anniversary (seed parent); Rosa Sky Tower (pollen parent)
The plant:
   Density.—medium to bushy.
   Growth type.—compact.
   Growth rate.—comparable to other roses.
   Height.—1.2 M.
   Width.—900 mm.
Leaf:
   Color.—UPPER: RHS 136A; Lower RHS 137B.
   Length.—90-110 mm.
   Width.—70-75 mm.
   Number.—3-5 leaflets.
   Shape.—ovate to orbicular, with serrated edge, pointed tip, and obtuse base.
Stem:
   Stem diameter.—average 10 mm, maximum 18 mm.
Inflorescence:
   Sepal.—5.
   Number of petals.—26-50.
   Petal size.—Medium.
   Petal width.—30-45 mm.
   Petal length.—36-42 mm.
   Petal arrangement.—Overlapping.
   Pedicel.—smooth (no prickles).
   Pedicel length.—50-70 mm.
   Pedicel color.—mid-green, upper surface brushed orange — brown.
   Bud shape.—ovate.
   Flower profile upper.—flattened.
   Flower profile lower.—convex.
   Time of flowering.—early.
   Length of flowering.—extended flowering.
   Color — midzone (outside).—RHS 155B.
   Color — midzone (inside).—RHS 155C.
   Color — margin.—white/near white.
   Color — basal spot.—soft lemon.
   Color — filament.—soft white with hint of lemon.
   Color — anther.—soft orange.
   Color — style.—white with hint of green.
Seed:
   Size.—medium.
   Color.—light green coloring to orange.
Botanical information: Flower polypetalous, ovary inferior, petals and stamen epigynous, receptacle globose to urn shaped. Multiple stamens arranged in whorls, multiple filaments in central column. Yellow pollen, sets seed producing a nLumber of small to medium sized hips of orange.

### GENERAL CHARACTERISTICS

A new rose variety with an upright, erect flowers and a compact, short bush includes white flowers with overlapping petals and trifoliate or simple compound leaves of dark green. The variety possesses a light but pleasing fragrance and exhibits very good disease resistance.

What is claimed is:

1. A new and distinct variety of Rose plant, substantially as shown and described.
